(19) United States
(12) Patent Application Publication
(10) Pub. No.: US 2012/0183546 A1
(43) Pub. Date: Jul. 19, 2012

(54) TREATMENT OF OVARIAN CANCER USING A SPECIFIC BINDING AGENT OF HUMAN ANGIOPOIETIN-2 IN COMBINATION WITH A TAXANE

(75) Inventor: David M. Weinreich, Simi Valley, CA (US)

(73) Assignee: AMGEN INC., Thousand Oaks, CA (US)

(21) Appl. No.: 13/497,774

(22) PCT Filed: Sep. 23, 2010
(86) PCT No.: PCT/US10/50033

§ 371 Date: Mar. 30, 2012

Related U.S. Application Data
(60) Provisional application No. 61/245,138, filed on Sep. 23, 2009.

(51) Int. Cl. A61K 39/395; A61P 35/00

(52) U.S. Cl. 424/134.1; 424/130.1

(57) ABSTRACT

Methods and compositions for treating ovarian cancer in a patient comprising administering a therapeutically effective amount of an Angiopoictin-2 inhibitor in combination with a taxane.

### FIELD OF THE INVENTION

The present invention relates to a method of treating ovarian cancer in a human patient by administering a therapeutically effective amount of an Ang2 inhibitor in combination with a taxane.

### BACKGROUND OF THE INVENTION

Angiogenesis, the formation of new blood vessels from existing ones, is essential to many physiological and pathological processes. Normally, angiogenesis is tightly regulated by pro- and anti-angiogenic factors, but in the case of diseases such as cancer, ocular neovascular diseases, arthritis, and psoriasis, the process can go awry. Folkman, J., Nat. Med., 1:27-31 (1995).

Although many signal transduction systems have been implicated in the regulation of angiogenesis, one of the best-characterized and most endothelial cell-selective systems involves the Tie2 receptor tyrosine kinase (NCBI Reference No. NP 000450.2; referred to as “Tie2” or “Tie2R” (also referred to as “ORK”); murine Tie2 is also referred to as “tek”) and its ligands, the angiopoietins (Gale, N. W. and Yancopoulos, G. D., Genes Dev. 13:1055-1066 [1999]). There are 4 known angiopoietins; angiopoietin-1 (“Ang1”) through angiopoietin-4 (“Ang4”). These angiopoietins are also referred to as “Tie2 ligands.

Numerous published studies have purportedly demonstrated vessel-selective Ang2 expression in disease states associated with angiogenesis. Most of these studies have focused on cancer, in which many tumor types appear to display vascular Ang2 expression. In contrast with its expression in pathological angiogenesis, Ang2 expression in normal tissues is extremely limited (Maisonpierre, P. C., et al., [1997], supra; Mezquita, J., et al., Biochemical and Biophysical Research Communications, 260:492-498 [1999]). In the normal adult, the three main sites of angiogenesis are the ovary, placenta, and uterus; these are the primary tissues in normal (i.e., non-cancerous) tissues in which Ang2 mRNA has been detected.

Ovarian cancer is the leading cause of death from a gynecologic cancer in the United States. In the United States, there were approximately 20,000 new cases and over 15,000 deaths attributable to ovarian cancer [Ozols, 2006]. In most cases, the high death rate is due to recurrence from a tumor that has spread beyond the ovary at the time of diagnosis. With modern surgical interventions and contemporary chemotherapy, most patients attain a temporary complete clinical remission. However, the majority will eventually have a relapse and die of complications of their disease.

An effective anti-Ang2 therapy would benefit a significant population of cancer patients because most solid tumors require neovascularization to grow beyond 1-2 millimeters in diameter. More specific to the present invention, such therapy might benefit patients with ovarian cancer. Accordingly, it is an object of the present invention to provide a method of inhibiting the growth of ovarian cancer in human patients.

### SUMMARY OF THE INVENTION

The present invention is directed in one embodiment to a method of treating ovarian cancer in a human patient by administering a therapeutically effective amount of an Ang2 inhibitor and/or a Tie2 inhibitor in combination with a taxane. In some embodiments the taxane is paclitaxel, docetaxel, or a derivative thereof. The Ang2 inhibitor of the present invention can be an antibody, Fc-peptide fusion protein (such as a peptibody), Fc-Tie2 extracellular domain (ECD) fusion protein (a “Tie2 trap”), or a small molecule inhibitor of Tie2.

### DETAILED DESCRIPTION

The present invention relates to compositions and methods for inhibiting progression of ovarian epithelial carcinomas in a human patient by administering a therapeutically effective amount of an Ang2 or Tie2 inhibitor in combination with a taxane, such as paclitaxel, docetaxel, or derivatives thereof.

The section headings are used herein for organizational purposes only, and are not to be construed as in any way limiting the subject matter described. The disclosure of all patents, patent applications, and other documents cited herein are hereby expressly incorporated by reference in their entirety. Unless specific definitions are provided, the nomenclature utilized in connection with, and the laboratory procedures and techniques of analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

### DEFINITIONS

The terms used throughout this specification are defined as follows, unless otherwise limited in specific instances.

The term “Ang2” refers to the polypeptide set forth in FIG. 6 of U.S. Pat. No. 6,166,185 (“Tie2 ligand-2”) as well as related native (i.e., wild-type) polypeptides such as allelic variants or splice variants (isoforms).

The term “Ang2 inhibitor” refers to an Ang2-specific binding agent that binds to human Ang2 inhibiting its binding to the human Tie2 receptor and resulting in a statistically significant decrease in angiogenesis, as measured by at least one functional assay of angiogenesis such as tumor endothelial cell proliferation or the corneal micropocket assay (See, Oliner et al. Cancer Cell 6:507-516, 2004). See also, U.S. Pat. Nos. 5,712,291 and 5,871,723. As those of ordinary skill in the art are aware, a corneal micropocket assay can be used to quantify the inhibition of angiogenesis. In this assay, agents to be tested for angiogenic activity are absorbed into a nylon membrane, which is implanted into micropockets created in the corneal epithelium of anesthetized mice or rats. Vascularization is measured as the number and extent of vessel ingrowth from the vascularized corneal limbus into the normally avascular cornea. See, U.S. Pat. No. 6,248,327 which describes planar migration and corneal pocket assays. In certain embodiments, the Ang2 inhibitor is an antibody, avimer (Nature Biotechnology 23, 1556-1561 (2005)), peptibody (Fc-peptide fusion protein), Fc-soluble Tie2 receptor fusion (i.e., a “Tie2 trap”), or small molecule Ang2 inhibitor.

The term “antibody” includes reference to isolated forms of both glycosylated and non-glycosylated immunoglobulins of any isotype or subclass, including any combination of: 1) human (e.g., CDR-grafted), humanized, and chimeric antibodies, and, 2) monospecific or multi-specific antibodies, monoclonal, polyclonal, or single chain (scFv) antibodies, irrespective of whether such antibodies are produced, in whole or in part, via immunization, through recombinant technology, by way of in vitro synthetic means, or otherwise. Thus, the term “antibody” is inclusive of those that are prepared, expressed, created or isolated by recombinant means, such as (a) antibodies isolated from an animal (e.g., a mouse) that is transgenic for human immunoglobulin genes or a hybridoma prepared therefrom, (b) antibodies isolated from a host cell transfected to express the antibody (e.g., from a transfectoma), (c) antibodies isolated from a recombinant, combinatorial antibody library, and (d) antibodies prepared, expressed, created or isolated by any other means that involve splicing of immunoglobulin gene sequences to other DNA sequences. In some embodiments the antibodies of the present invention are monoclonal antibodies, such as humanized or fully-human monoclonal antibodies. Typically, antibodies of the present invention will be IgG1 or IgG2 subclass antibodies. The antibody may bind Ang2 or Tie2 with a Kd of less than about 10 nM, 5 nM, 1 nM, or 500 μM.

The terms “derivation” or “derivatives” generally refer to modification of an Ang2 or Tic2 inhibitor, or of a taxanc such as paclitaxel or docetaxel, by covalently linking it, directly or indirectly, so as to modify such characteristics as half-life, bioavailability, immunogenicity, solubility, or hypersensitivity, while retaining its therapeutic benefit. Derivatives can be made by glycosylation, pegylation, and lipidation, or by protein conjugation of an Ang2 inhibitor, Tie2 inhibitor, or a taxane (e.g., paclitaxel, docetaxel) and are within the scope of the present invention. Exemplary derivitizing agents include an Fc domain as well as a linear polymer (e.g., polyethylene glycol (PEG), polylysine, dextran, etc.); a branched-chain polymer (See, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993); a lipid or liposome; a cholesterol group (such as a steroid); a carbohydrate or oligosaccharide.

The terms “effective amount” or “therapeutically effective amount” when used in relation to an Ang2 or Tie2 inhibitor refers to an amount that when used in a combination therapy with a taxane (e.g., paclitaxel, docetaxel, or derivatives thereof) yields a statistically significant inhibition of ovarian cancer progression in an ovarian cancer patient population of statistically significant size relative to treatment with the Ang2 inhibitor or Tie2 inhibitor alone or the taxane alone. As used herein, the terms “treatment”, “treating”, “inhibiting” or “inhibition” of ovarian cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in survival relative to treatment with a taxane (e.g., paclitaxel or docetaxel) alone.

The term “Fc” in the context of an antibody or peptibody of the present invention is typically fully human Fc, and may be any of the immunoglobulins, although IgG1 and IgG2 are preferred. However, Fc molecules that are partially human, or obtained from non-human species are also included herein.

The term “Fc-peptide fusion” refers to a peptide that is covalently bonded, directly or indirectly, to an Fc. Exemplary Fc-peptide fusion molecules include a peptibody such as those disclosed in WO 03/057134, incorporated herein by reference, as well as an Fc covalently bonded, directly or indirectly, to an Ang2 specific binding fragment of the Tie2 receptor.

The term “host cell” refers to a cell that can be used to express a nucleic acid. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include the COS-7 line of monkey kidney cells (ATCC CRL 1651) (see Gluzman et al., Cell 23: 175, 1981), L cells, C127 cells, 3T3 cells (ATCC CCL 163), Chinese hamster ovary (CHO) cells or their derivatives such as Veggie CHO and related cell lines which grow in serum-free media (see Rasmussen et al., Cytotechnology 28: 31, 1998) or CHO strain DX-B11, which is deficient in DHFR (see Urlaub et al., Proc. Natl. Acad. Sci. USA 77: 4216-4220, 1980).

The term “human antibody” refers to an antibody in which both the constant regions and the framework consist of fully or substantially human sequences such that the human antibody elicits substantially no immunogenic reaction against itself when administered to a human host and preferably, no detectable immunogenic reaction.

The term “humanized antibody” refers to an antibody in which substantially all of the constant region is derived from or corresponds to human immunoglobulins, while all or part of one or more variable regions is derived from another species, for example a mouse.

The term “isolated” refers to a compound that is: (1) is substantially purified (e.g., at least 60%, 70%, 80%, or 90%) away from cellular components with which it is admixed in its expressed state such that it is the predominant species present, (2) is conjugated to a polypeptide or other moiety to which it is not linked in nature, (3) does not occur in nature as part of a larger polypeptide sequence, (4) is combined with other chemical or biological agents having different specificities in a well-defined composition, or (5) comprises a human engineered sequence not otherwise found in nature.

The terms “monoclonal antibody” or “monoclonal antibody composition” refers to a preparation of antibody molecules of single molecular composition, typically encoded by identical nucleic acid molecules. A monoclonal antibody composition displays a single binding specificity and affinity for a particular epitope. In certain embodiments, monoclonal antibodies are produced by a single hybridoma or other cell line (e.g., a transfectoma), or by a transgenic mammal. The term “monoclonal” is not limited to any particular method for making an antibody.

The terms “naturally occurring” or “native” when used in connection with biological materials such as nucleic acid molecules, polypeptides, host cells, and the like, refers to those which are found in nature and not modified by a human being.

The terms, “nucleic acid” and “polynucleotide” refer to a deoxyribonucleotide or ribonucleotide polymer, or chimeras thereof, and unless otherwise limited, encompasses the complementary strand of the referenced sequence. A nucleic acid sequence is “operably linked” to a regulatory sequence if the regulatory sequence affects the expression (e.g., the level, timing, or location of expression) of the nucleic sequence. A “regulatory sequence” is a nucleic acid that affects the expression (e.g., the level, timing, or location of expression) of a second nucleic acid. Thus, a regulatory sequence and a second sequence are operably linked if a functional linkage between the regulatory sequence and the second sequence is such that the regulatory sequence initiates and mediates transcription of the DNA sequence corresponding to the second sequence. Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., Nucleic Acids Res. 23: 3605-3606, 1995.

The terms “peptide,” “polypeptide” and “protein” are used interchangeably throughout and refer to a molecule comprising two or more amino acid residues joined to each other by peptide bonds. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation.

The term “peptibody” refers to a specific binding agent that is a molecule comprising an antibody Fc domain attached to at least one peptide. The production of peptibodies is generally described in PCT publication WO 00/24782, published May 4, 2000, incorporated herein by reference. Exemplary peptides may be generated by any of the methods set forth therein, such as carried in a peptide library (e.g., a phage display library), generated by chemical synthesis, derived by digestion of proteins, or generated using recombinant DNA techniques.

The terms “peptibody fragment” or “antibody fragment” refers to a peptide or polypeptide of a peptibody or antibody which comprises less than a complete intact antibody or peptibody but retains the ability to specifically bind to its target molecule (e.g., Ang2). Exemplary fragments includes F(ab) or F(ab)₂ fragments. Such a fragment may arise, for example, from a truncation at the amino terminus, a truncation at the carboxy-terminus, and/or an internal deletion of a residue(s) from the amino acid sequence. Fragments may result from alternative RNA splicing or from in vivo or in vitro protease activity. Such fragments may also be constructed by chemical peptide synthesis methods, or by modifying a polynucleotide encoding an antibody or peptibody.

The terms “polynucleotide,” “oligonucleotide” and “nucleic acid” are used interchangeably throughout and include DNA molecules (e.g., cDNA or genomic DNA), RNA molecules (e.g., mRNA), and hybrids thereof. The nucleic acid molecule can be single-stranded or double-stranded.

The term “specific binding agent” refers to an Ang2 inhibitor or Tie2 inhibitor. A specific binding agent may be a protein, peptide, nucleic acid, carbohydrate, lipid, or small molecular weight compound that specifically binds to Ang2 or Tie2. In a preferred embodiment, the specific binding agent according to the present invention is an antibody or binding fragment thereof (e.g., Fab, F(ab′)2), peptide or a peptibody, Ang2 binding fragments thereof, or Fc-Tie2 extracellular domain (ECD) fusion protein (“Tie2 trap”). WO00/24782 and WO03/057134 (incorporated herein by reference) describe and teach making binding agents that contain a randomly generated peptide which binds a desired target. A specific binding agent can be a proteinaceous polymeric molecule (a “large molecule”) such as an antibody or Fc-peptide fusion, or a non-proteinaceous non-polymeric molecule typically having a molecular weight of less than about 1200 Daltons (a “small molecule”).

The term “specifically binds” refers to the ability of a specific binding agent of the present invention, under specific binding conditions, to bind a target molecule such that its affinity is at least 10 times as great, but optionally 50 times as great, 100, 250 or 500 times as great, or even at least 1000 times as great as the average affinity of the same specific binding agent to a large collection of random peptides or polypeptides. A specific binding agent need not bind exclusively to a single target molecule but may specifically bind to a non-target molecule due to similarity in structural conformation between the target and non-target (e.g., paralogs or orthologs). Those of skill will recognize that specific binding to a molecule having the same function in a different species of animal (i.e., ortholog) or to a molecule having a substantially similar epitope as the target molecule (e.g., a paralog) is within the scope of the term “specific binding” which is determined relative to a statistically valid sampling of unique non-targets (e.g., random polypeptides). Thus, a specific binding agent of the invention may specifically bind to more than one distinct species of target molecule, such as specifically binding to both Ang2 and Ang1. Solid-phase ELISA immunoassays can be used to determine specific binding. Generally, specific binding proceeds with an association constant of at least about 1×10⁷ M⁻¹, and often at least 1×10⁸M⁻¹, 1×10⁹ M⁻¹, or, 1×10¹⁰ M⁻¹.

The term “Tie2 inhibitor” refers to a Tie2 specific binding agent that binds to human Tie2 and inhibits its binding to Ang2 and/or inhibits Tie2 signal transduction and resulting in a statistically significant decrease in angiogenesis, as measured by at least one functional assay of angiogenesis such as tumor endothelial cell proliferation or the corneal micropocket assay (Oliner et al. Cancer Cell 6:507-516, 2004). See also, U.S. Pat. Nos. 5,712,291 and 5,871,723 (both incorporated herein by reference). In certain embodiments, the Tie2 inhibitor is an antibody, avimer (Nature Biotechnology 23, 1556-1561 (2005)), peptibody, or small molecule Ang2 inhibitor.

The term “vector” refers to a nucleic acid used in the introduction of a polynucleotide of the present invention into a host cell. Vectors are often replicons. Expression vectors permit transcription of a nucleic acid inserted therein when present in a suitable host cell or under suitable in vitro conditions.

### Combination Therapy for Treatment of Ovarian Cancer

The present invention is directed to a method of treating ovarian epithelial carcinomas in a human patient with a specific binding agent so as to inhibit, halt, reverse progression of the tumor, or otherwise result in a statistically significant increase in progression-free survival (i.e., the length of time during and after treatment in which a patient is living with ovarian cancer that does not get worse), or overall survival (also called “survival rate”; i.e., the percentage of people in a study or treatment group who are alive for a certain period of time after they were diagnosed with or treated for ovarian cancer) relative to treatment with a taxane (such as, but not limited to, paclitaxel or docetaxel) alone or a specific binding agent alone. The method comprises administering to the patient a therapeutically effective amount of an Ang2 and/or Tie2 inhibitor in combination with a taxane. In some embodiments, the patient is refractory to platinum based chemotherapy for ovarian cancer. Exemplary platinum based chemotherapeutics include cisplatin, carboplatin, and oxaliplatin.

The Ang2 and Tie2 inhibitors of the present invention are specific binding agents that inhibit interaction between Ang2 and its receptor Tie2 and/or inhibit Tie2 signal transduction thereby inhibiting tumor angiogenesis. In some embodiments, the Ang2 inhibitor also specifically binds to Ang1 and inhibits Ang1 binding to the Tie2 receptor (a “dual Ang2 and Ang1 inhibitor”). The Ang2 and Tie2 inhibitors are inclusive of large molecules such as a peptide, peptibody, antibody, antibody binding fragment such as a F(ab) or F(ab′)2 fragment, an Fc-Tie2 extracellular domain (ECD) fusion protein (a “Tie2 trap”), and small molecules, or combinations thereof. In specific embodiments, the specific binding agent is an Ang2 inhibitory peptibody as discussed in more detail infra.

In the method of the present invention, a therapeutically effective amount of the Ang2 or Tie2 inhibitor is administered in combination with a taxane as a chemotherapeutic agent. The therapeutically effective dose of the specific binding agent can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

The frequency of dosing will depend upon the pharmacokinetic parameters of the binding agent molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The specific binding agent is administered at doses and rates readily determined by those of ordinary skill in the art. In some embodiments, the specific binding agent is an antibody or peptibody administered intravenously once a week. In some embodiments, the taxane is administered once a week (e.g., intravenously) for three weeks and is not administered the fourth week of a four week cycle. In some embodiments, the Ang2 or Tie2 inhibitor of the present invention is administered to the patient at a dose ranging from 0.3 to 30 mg/kg of patient body weight, often at from 1 to 20 mg/kg, or 3 to 15 mg/kg. In certain embodiments the taxane (e.g., paclitaxel) is administered once a week at approximately 40 to 120 mg/m² (square meter of patient surface area), often at between 50 to 80 mg/m2, and in some specific embodiments at around 80 mg/m2. In other embodiments, when administered once every three weeks, the dose of taxane ranges from between 50 to 225 mg/m2, often at 135 to 200 mg/m2. Conveniently, the dose of Ang2 inhibitor of the present invention is calculated using the standard pharmacokinetic parameter AUC (area under curve) wherein the dosage range is 5-40 mg-hr/ml, often at 6 to 25 mg-hr/ml. In some embodiments, the dose in milligrams of peptibody 2XCon4(C) to be administered is calculated per the formula 530+(5.0*Baseline Creatinine Clearance [in mL/min]), wherein baseline creatinine clearance (CrCL) is to be determined by the Cockcroft and Gault equation (Nephron 1976 16: 31-41). Thus, when the CrCL is between 40 to 70 the calculated dose is 840 mg of 2XCon4(C), when the CrCL is 70 to 90 the calculated dose is 960 mg, when the CrCL is 90 to 110 the calculated dose is 1080 mg, when the CrCL is 110 to 140 the calculated dose is 1200 mg, when the CrCL is 140 to 160 the calculated dose is 1320 mg, when the CrCL is 160 to 190 the calculated dose is 1440 mg, when the CrCL is 190 to 200 the calculated dose is 1560 mg of 2XCon4(C).

The taxane of the present invention can be administered prior to and/or subsequent to (collectively, “sequential treatment”), and/or simultaneously with (“concurrent treatment”) a specific binding agent of the present invention. Sequential treatment (such as pretreatment, post-treatment, or overlapping treatment) of the combination, also includes regimens in which the drugs are alternated, or wherein one component is administered long-term and the other(s) are administered intermittently. Components of the combination may be administered in the same or in separate compositions, and by the same or different routes of administration. Methods and dosing of administering chemotherapeutic agents are known in the art. Standard dosages and methods of administrations can be used, for example per the Food and Drug Administration (FDA) label. The taxane of the present invention may be given as a drip (infusion) through a cannula inserted into a vein (IV), through a central line, which is inserted under the skin into a vein near the collarbone, or a peripherally inserted central catheter (PICC) line. The dose of taxanc is often administered in a fixed-time such as 30 minutes. Alternatively, the dose can be administered at a fixed rate (e.g., 10 mg/m²/minute).

### Specific Binding Agents

Specific binding agents of the present invention are known in the art or may be prepared using methods known in the art. Exemplary Ang2 specific binding agents are taught and disclosed in U.S. Pat. No. 7,138,370 (Oliner et al.); PCT WO 2006/068953 (Green et al.); U.S. Ser. No. 12/378,993 filed on Feb. 19, 2009 (Oliner et al.); PCT WO 2006/045049 (Oliner et al.), all of which are incorporated herein by reference. In a specific embodiment, the specific binding agent is 2XCon4(C) (alternatively referred to as AMG 386), a dual Ang2 and Ang1 inhibitor, as disclosed in U.S. Pat. No. 7,138,370 at SEQ ID NO: 25 (herein as SEQ ID NO: 1), wherein the Fc of SEQ ID NO: 25 is an IgG1 Fc such as that disclosed in SEQ ID NO: 60 (herein as SEQ ID NO: 2). In another embodiment the specific binding agent is antibody H4L4 disclosed in U.S. Ser. No. 12/378,993 (incorporated herein by reference) wherein the heavy chain has the sequence of SEQ ID NO: 3 of U.S. Ser. No. 12/378,993 (herein as SEQ ID NO: 3), and wherein the light chain has the sequence of SEQ ID NO: 10 of U.S. Ser. No. 12/378,993 (herein as SEQ ID NO: 4).

Exemplary Ang2 and Tic2 large molecule and small molecule inhibitors included within the scope of the present invention include: PF-4856884 (CovX 60; Pfizer), AP-25434 (Ariad), ARRY-614 (Array), CE-245677 (Pfizer), CEP-11981 (Cephalon), SSR-106462 (Sanofi), MGCD-265 (Methylgene), CGI-1842 (CGI Pharma, Genentech), CGEN-25017 (Compugen), DX-2240 (Dyax, Sanofi), MEDI3617 (MedImmune), Antibody 3.19.3 (Astra Zeneca), and LP-590 (Locus Pharmaceuticals). In specific embodiments the taxane of the present invention is paclitaxel (TAXOL), docetaxel (TAXOTERE), or taxane derivatives such as ABRAXANE (albumin-bound paclitaxel), PG-paclitaxel or DHA-paclitaxel.

In general, specific binding agents such as antibodies, antibody fragments, peptibodies, avimers, or Fc-peptide fusions, that specifically bind and inhibit Tie2, Ang2, or Ang1 and Ang2 polypeptides are within the scope of the present invention. The antibodies may be isolated polyclonal or monoclonal (mAbs). The polyclonal or monoclonal antibodies can be chimeric, humanized such as CDR-grafted, fully human, single chain, bispecific, as well as antigen-binding fragments and/or derivatives thereof.

Monoclonal antibodies specifically binding to and functioning as an Ang2, a dual Ang2 and Ang1, or a Tie2 inhibitor can be produced using, for example but without limitation, the traditional “hybridoma” method or the newer “phage display” technique. For example, monoclonal antibodies of the invention may be made by the hybridoma method as described in Kohler et al., Nature 256:495 [1975]; the human B-cell hybridoma technique [Kosbor et al., Immunol Today 4:72 (1983); Cote et al., Proc Natl Acad Sci (USA) 80: 2026-2030 (1983); Brodeur et al., Monoclonal Antibody Production Techniques and Applications, pp. 51-63, Marcel Dekker, Inc., New York, (1987)] and the EBV-hybridoma technique [Cole et al., Monoclonal Antibodies and Cancer Therapy, Alan R Liss Inc, New York N.Y., pp 77-96, (1985)].

The phage display technique may also be used to generate monoclonal antibodies. Preferably, this technique is used to produce fully human monoclonal antibodies in which a polynucleotide encoding a single Fab or Fv antibody fragment is expressed on the surface of a phage particle. [Hoogenboom et al., J Mol Biol 227: 381 (1991); Marks et al., J Mol Biol 222: 581 (1991); see also U.S. Pat. No. 5,885,793)]. Each phage can be “screened” using standard binding and cell-based assays to identify those antibody fragments having affinity for, and inhibition of, Ang2 or Tie2. Once polynucleotide sequences are identified which encode each chain of the full length monoclonal antibody or the Fab or Fv fragment(s) of the invention, host cells, either eukaryotic or prokaryotic, may be used to express the monoclonal antibody polynucleotides using recombinant techniques well known and routinely practiced in the art.

In another embodiment of the present invention, a monoclonal or polyclonal antibody or fragment thereof that is derived from other than a human species may be “humanized” or “chimerized”. Methods for humanizing non-human antibodies are well known in the art. (see U.S. Pat. Nos. 5,859,205, 5,585,089, and 5,693,762). Humanization is performed, for example, using methods described in the art [Jones et al., Nature 321: 522-525 (1986); Riechmann et al., Nature, 332: 323-327 (1988); Verhoeyen et al., Science 239:1534-1536 (1988)] by substituting at least a portion of, for example a rodent, complementarity-determining region (CDRs) for the corresponding regions of a human antibody.

Alternatively, transgenic animals (e.g., mice) that are capable of producing a repertoire of human antibodies in the absence of endogenous immunoglobulin production can be used to generate such antibodies. This can be accomplished by immunization of the animal with an Ang2 or Tie2 antigen or fragments thereof (e.g., the Tie2 extracellular domain). Such immunogens can be optionally conjugated to a carrier. See, for example, Jakobovits et al., Proc Natl Acad Sci (USA), 90: 2551-2555 (1993); Jakobovits et al., Nature 362: 255-258 (1993); Bruggermann et al., Year in Immuno, 7: 33 (1993). In one method, such transgenic animals are produced by incapacitating the endogenous loci encoding the heavy and light immunoglobulin chains therein, and inserting loci encoding human heavy and light chain proteins into the genome thereof. Partially modified animals, that are those having less than the full complement of these modifications, are then crossbred to obtain an animal having all of the desired immune system modifications. When administered an immunogen, these transgenic animals are capable of producing antibodies with human variable regions, including human (rather than e.g., murine) amino acid sequences, that are immuno-specific for the desired antigens. See PCT application Nos., PCT/US96/05928 and PCT/US93/06926. Additional methods are described in U.S. Pat. No. 5,545,807, PCT application Nos. PCT/US91/245, PCT/GB89/01207, and in EP 546073B1 and EP 546073A1. Human antibodies may also be produced by the expression of recombinant DNA in host cells or by expression in hybridoma cells as described herein.

Large-scale production of chimeric, humanized, CDR-grafted, and fully human antibodies, or antigen-binding fragments thereof, are typically produced by recombinant methods. Polynucleotide molecule(s) encoding the heavy and light chains of each antibody or antigen-binding fragments thereof, can be introduced into host cells and expressed using materials and procedures described herein. In a particular embodiment, the antibodies are produced in mammalian host cells, such as CHO cells.

### Pharmaceutical Formulation

The pharmaceutical composition comprising the Ang2 and/or Tie2 inhibitors of the present invention may contain formulation materials for modifying, maintaining or preserving, for example, the pH, osmolarity, viscosity, clarity, color, isotonicity, odor, sterility, stability, rate of dissolution or release, adsorption, or penetration of the composition.

The primary vehicle or carrier in a pharmaceutical composition may be either aqueous or non-aqueous in nature. For example, a suitable vehicle or carrier may be water for injection or physiological saline, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefore. In one embodiment of the present invention, binding agent compositions may be prepared for storage by mixing the selected composition having the desired degree of purity with optional formulation agents (Remington's Pharmaceutical Sciences, supra) in the form of a lyophilized cake or an aqueous solution. Further, the binding agent product may be formulated as a lyophilizate using appropriate excipients such as sucrose.

The formulation components are present in concentrations that are acceptable to the site of administration. For example, buffers are used to maintain the composition at physiological pH or at slightly lower pH, typically within a pH range of from about 5 to about 8. A particularly suitable vehicle for parenteral administration is sterile distilled water in which a binding agent is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provide for the controlled or sustained release of the product which may then be delivered via a depot injection.

In another aspect, pharmaceutical formulations suitable for parenteral administration may be formulated in aqueous solutions, preferably in physiologically compatible buffers such as Hanks' solution, ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration. In a specific embodiment, a lyophilized peptibody, such as 2XCon4(C), is formulated as disclosed in WO 2007/124090 (incorporated herein by reference).

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding agent molecule is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 50 mg/kg or more, depending on the factors mentioned above. In other embodiments, the dosage may range from 0.1 mg/kg up to about 100 mg/kg; or 1 mg/kg up to about 100 mg/kg; or 5 mg/kg up to about 50 mg/kg.

The above listings are by way of example only, and do not preclude the use of other compounds or treatments which can be used concurrently with the compounds described herein that are known by those skilled in the art or that could be arrived at by those skilled in the art using the guidelines set forth in this specification.

### Example 1

Example 1 describes the safety, pharmacokinetics, and anti-tumor activity of AMG 386 (2XCon4(C)), a selective angiopoietin inhibitor, in adult patients with advanced solid tumors.

Institutional Review Board approval and written informed consents were obtained before study-related procedures were started. Inclusion criteria included age≧18 years; histologically documented advanced solid tumors refractory to standard treatment for which no standard therapy was available or for patients who refused standard treatment; Eastern Cooperative Oncology Group (ECOG) performance status≦2; and adequate hematologic, hepatic, and renal function. Exclusion criteria included: CNS (Central Nervous System) metastasis; leukemia or myelodysplastic syndrome; unstable angina; congestive heart failure (New York Health Association class>II); uncontrolled hypertension (diastolic>85 mmHg; systolic>145 mmHg); cardiac arrhythmia; coagulation disorders (hypercoagulopathy, bleeding diathesis, or condition requiring therapeutic anticoagulation); and anti-tumor treatment within 3 weeks of study day 1 (within 6 weeks for antibody therapy).

### Study Design

This was a first-in-human, phase 1, open-label study with a dose-escalation and a dose-expansion phase. AMG 386 was administered as a 60-minute (later reduced to a 30-minute) intravenous (IV) infusion once every week (QW). The starting dose was calculated based on the 28-day toxicology study in rats. In the dose-escalation phase, patients were enrolled sequentially into 5 dose cohorts (0.3, 1, 3, 10, or 30 mg/kg; 4-6 patients per cohort). Initially, 4 patients received AMG 386 IV QW for up to 4 weeks, and dose escalation proceeded if no dose-limiting toxicity (DLT) was observed in the first 4 weeks. If 1 patient experienced a DLT, up to 2 additional patients were to have been enrolled at that dose level. After 4 weeks of treatment, tumor response was evaluated by computed tomography (CT) or magnetic resonance imaging (MRI) per Response Evaluation Criteria in Solid Tumors (RECIST). In patients without progressive disease (PD), AMG 386 was resumed at week 6 (AMG 386 was withheld at week 5 for evaluation of terminal PK parameters). Ten patients entered the dose-expansion phase (30-mg/kg AMG 386 IV QW) to obtain additional safety and PK data and to explore potential anti-tumor activity. Patients remained on study until disease progression, unacceptable toxicities, or withdrawal of consent.

### Definition of DLT and Maximum Tolerated Dose (MTD)

DLT was defined as any treatment-related, grade≧3 toxicity (according to Common Terminology Criteria for Adverse Events [CTCAE], version 3.0; excluding grade 3 transient infusion reactions) during the first 4 weeks. MTD was defined as the highest dose with an observed incidence of a DLT in <33% of patients per cohort.

### Dose Adjustments

If patients experienced grade 3 or 4 toxicities or serious toxicities not considered related to AMG 386, treatment was postponed until the event resolved to grade 1 or mild, or returned to the patient's baseline value. No intra-patient dose adjustment was allowed until the MTD was established or all patients in the 30-mg/kg cohort completed 4 weeks of treatment without significant safety signals. Patients enrolled at lower dose levels could then receive 30-mg/kg AMG 386.

### Evaluation of Safety

Toxicities (graded using CTCAE) were recorded for patients who received≧1 dose of AMG 386. Blood pressure was measured before and after every infusion and 1, 2, 6, 21, 48, and 96 h after the week 1 infusion. Urine protein was measured via dipstick during week 1 (predose, 24 and 48 h after the first dose), and every week thereafter (predose). AMG 386 is a recombinant Fc-peptide fusion protein and is potentially immunogenic in humans. Therefore, serum for assessment of anti-AMG 386 antibodies was collected pre-dose, at weeks 1, 2, 4, and 6, every 4 weeks thereafter, and approximately 4 weeks after the last dose of AMG 386. Anti-AMG 386 binding and neutralizing antibodies were assayed using an electrochemiluminescent immunoassay and a receptor-binding bioassay, respectively.

Pharmacokinetics (PK)

Serum samples for PK parameters were collected on day 1 and at week 4 pre-dose, immediately after infusion, and 2, 6, 24, 48, and 96 h after infusion. Two additional samples were collected at week 4, 168 and 264 h after infusion. Pre-dose PK serum samples were also collected at week 2, week 3, week 6, QW thereafter, and 4 weeks after the end-of-study visit. AMG 386 concentration was determined using an enzyme-linked immunosorbent assay. Serum PK parameters were estimated using noncompartmental methods with WinNonlin Professional (version 4.1e, Pharsight Corp., Mountain View, Calif., USA).

### Evaluation of Tumor Response

Tumors were evaluated using CT (computed tomography) or MR1 (magnetic resonance imaging) per RECIST criteria. For the dose-escalation and dose-expansion phases, evaluations were performed at week 4 and week 8, respectively, and every 8 weeks thereafter.

### Measurement of Pharmacodynamic Effects of AMG 386

Since AMG 386 inhibited tumor endothelial cell proliferation in preclinical experiments, we investigated whether AMG 386 could impact tumor vascularity in patients using dynamic contrast-enhanced (DCE)-MRI (3D-image acquisition). DCE-MRI was required only for patients in the dose-expansion phase (must have ≧1 tumor>3 cm outside the thoracic cavity) and was optional for those in the dose-escalation phase. DCE-MRI was performed within 5 days before the first administration of AMG 386, 48 h after the first dose, and either 48 h after the week 4 dose (dose-escalation) or the week 8 dose (dose-expansion) or both. Images were analyzed at an independent central image-analysis vendor (VirtualScopics, Inc, Rochester, N.Y.). Percent change from baseline in volume transfer constant (K^{trans}) determined using a data-derived input function and blood-normalized (first 90 seconds) area-under-the-enhancement curve (IAUC) were recorded for each tumor. These parameters reflect contrast delivery (capillary blood flow) and transport across the vascular endothelium (capillary permeability-surface area product), with the dominant factor depending on whether delivery is limited by flow or permeability.

### Statistical Analysis

Descriptive statistics are provided for demographic, safety, PK, and anti-tumor activity. Categorical data were summarized using frequency and percentages; continuous data were summarized by means, median, range, and coefficient of variation±standard deviation. A one sample t-test was used to test if the percent change from baseline in K^{trans} or IAUC differed from zero following AMG 386 administration.

### Patient Characteristics and Disposition

Thirty-two patients were enrolled between 10 Jan. 2005 and 31 May 2006. Four patients were enrolled into each of five planned dose levels. No DLTs were reported in patients receiving 0.3, 1, 3, or 10 mg/kg. A DLT was reported in 1 of 16 patients receiving 30 mg/kg. One patient received 31 doses of 3-mg/kg AMG 386 then 18 doses of 30-mg/kg AMG 386. Patients received a median of 6 doses of AMG 386. At the time of this analysis (26 Apr. 2007), 31 patients had discontinued the study due to disease progression (confirmed by radiography, n=17), clinical disease progression (investigator determined, n=6), protocol-specified criteria (n=3), toxicities (n=2), death (n=2), or withdrawal of consent (n=1). One patient with a partial response (PR) withdrew from the study after 156 weeks of treatment. Three patients in the 0.3-mg/kg cohort did not have a PR or complete response (CR) by week 4. The protocol was later amended to allow patients with stable disease (SD) at week 4 to continue treatment with AMG 386.

### DLT and MTD

AMG 386 appeared to be well tolerated at all doses. An MTD was not reached. One DLT was seen in a patient with carcinoma of unknown origin and extensive metastases to the liver, lungs, abdominal lymph nodes, and omentum who received 2 doses of 30-mg/kg AMG 386 and died on study day 11. Cause of death was attributed to respiratory failure due to tumor burden, but a potential contribution of AMG 386 could not be excluded.

### Safety

No dose-related trends were observed in the incidence or severity of toxicities. Toxicities reported in ≧3 patients were mostly grade 1 or 2. The most common (≧5 patients) toxicities were fatigue, peripheral edema, insomnia, upper abdominal pain, back pain, and nausea. Two patients had toxicities leading to discontinuation of AMG 386: 1 with nephrolithiasis (10-mg/kg cohort) and 1 with intestinal obstruction (30-mg/kg cohort); neither was considered related to AMG 386. Treatment-related toxicities were reported for 16 (50%) patients, and, except for the death due to respiratory arrest, were grade 1 or 2. The most common treatment-related toxicities were fatigue (n=8) and peripheral edema (n=4). Six patients had ≧1 toxicities that met the definition of a serious adverse event; of these, only one was considered possibly attributable to AMG 386 (see DLT above). The others were not attributed to AMG 386: 1 patient died of respiratory failure due to progressive disease (patient had end-stage sarcoma with pleural effusions and ascites and received 4 doses of 1-mg/kg AMG 386): 1 had grade 3 recurrent pleural effusions (3-mg/kg cohort); 1 had grade 3 gastric outlet obstruction (3-mg/kg cohort); 1 had grade 3 catheter-related infection (30-mg/kg cohort); and 1 had grade 3 bowel obstruction and abdominal pain and grade 2 pulmonary edema (30-mg/kg cohort).

Compared with toxicities expected from anti-VEGF therapies, no bleeding or thromboembolic events were observed. Transient grade 1 or 2 hypertension was noted in 2 patients but was not attributed to AMG 386; both patients had pre-existing hypertension and were receiving anti-hypertensive medications before and during the study. Proteinuria reported in 11 patients (6 with grade≦2; 5 with grade 3) was transient (1-2 weeks and resolved without medical intervention) and did not result in any clinical sequelae. No patient discontinued AMG 386 due to proteinuria or hypertension. There were no clinically significant changes in serum chemistry or hematology laboratory values.

Two patients (6%) developed anti-AMG 386 binding antibodies during treatment that were transient for one patient; subsequent samples from the second patient were not available for analysis. One patient tested positive for pre-existing binding antibodies but was negative at subsequent time points, including the end-of-study sample. No patient developed anti-AMG 386 neutralizing antibodies. No infusion reactions were reported, and no infusions were interrupted or stopped as a result of toxicities.

### Pharmacokinetics

After 4 weekly IV infusions of AMG 386, serum AMG 386 levels appeared to increase proportionally to the dose administered, AMG 386 appeared to reach steady state, and minimal accumulation was observed. After the week 4 dose, the mean serum clearance appeared to be similar across each dose (overall mean, 1.17 mL/hr/kg) suggesting that AMG 386 exhibited linear PK. The mean terminal half-life was 3.1-6.3 days. At doses of AMG 386≧3 mg/kg, the average steady-state serum Cₘᵢₙ values were higher than those achieved with the optimal biological dose for inhibition of tumor growth in a preclinical tumor xenograft model (0.6 mg/kg subcutaneously twice weekly yielded a scrum Cₘᵢₙ of 3 μg/mL). Post-treatment anti-AMG 386 binding antibodies in 2 patients did not appear to affect AMG 386 exposure.

### Anti-tumor Activity

Of 29 patients evaluable for tumor response, 1 had a PR, 16 had a best response of SD, and 12 had PD. Four patients had SD for ≧16 weeks: 1 soft tissue sarcoma (191 days); 1 thyroid cancer (135 days); 1 pseudomyxoma (246 days); and 1 adenocarcinoma of the submandibular gland (247 days). Four patients demonstrated some reduction in tumor burden, 2 had a reduction>10%. The greatest tumor reduction (32.5%) was observed in a 76-year-old woman with advanced, refractory (to platinum based chemotherapy) ovarian cancer who received 30-mg/kg AMG 386 and achieved a PR at week 68, confirmed at week 72; she withdrew from the study with a PR after 156 weeks of treatment. Of note, this patient's serum CA-125 was 217 U/mL at baseline, 73 U/mL after 4 weeks of AMG 386 treatment, and remained between 20-40 U/mL for >2 years.

### Example 2

Example 2 is a description of a randomized, double-blind, placebo controlled, phase 2 trial of paclitaxel in combination with AMG 386 (2XCon4(C)) in subjects with advanced recurrent epithelial ovarian or primary peritoneal cancer. Additional details of this study can be found at the US National Institutes of Health website (clinicaltrials.gov), under study identifier: NCT00479817 (incorporated herein by reference).

The primary objective of the study is to estimate the treatment effect as measured by progression free survival (PFS) of subjects with recurrent ovarian cancer receiving AMG 386 (either 3 mg/kg or 10 mg/kg IV (intravenous) QW (once per week) in combination with paclitaxel (80 mg/m² IV QW; 3 on/1 off) compared to subjects receiving paclitaxel (80 mg/m² IV QW; 3 on/1 off) plus placebo. The study was designed as a phase 2, randomized, double-blind, placebo controlled, multi-center study to estimate the improvement in progression free survival (PFS) (compared to control subjects) and evaluate the safety and tolerability of AMG 386 in combination with paclitaxel in the treatment of subjects with advanced recurrent ovarian cancer. Subjects are to be randomized 1:1:1 ratio to each of the following arms:

Arm A: Paclitaxel 80 mg/m2 IV QW (3 weeks on/1 week off)+AMG 386 10 mg/kg IV QW
Arm B: Paclitaxel 80 mg/m2 IV QW (3 weeks on/1 week off)+AMG 386 3 mg/kg IV QW
Arm C: Paclitaxel 80 mg/m2 IV QW (3 weeks on/1 week off)+AMG 386 placebo.

To maintain the double-blind each subject was infused weekly with a volume of investigational product equivalent to 10 mg/kg AMG 386 IV. Subjects are discontinued from study treatment at any time for disease progression, clinical progression, unacceptable toxicity, subject withdrawal of consent, or death. Subjects on Arm C may receive open label AMG 386 (10 mg/kg IV QW) monotherapy if they meet certain pre-study eligibility requirements after disease progression per modified RECIST or clinical progression. The control group is defined as subjects who are randomized to Arm C: paclitaxel 80 mg/m² IV QW (3 on/1 off) and AMG 386 placebo.

Radiological imaging to assess disease status is performed every 8 weeks±7 days (2 cycles) during the study until subjects develop radiographic disease progression per the modified RECIST criteria. In addition, any subject who discontinues study drug treatment prior to radiographic disease progression per modified RECIST criteria will continue to have radiological imaging and CA-125 assessment performed every 8 weeks±7 days during the long term follow up period until the subject develops radiographic disease progression or begins a new treatment. The primary endpoint is progression free survival (PFS).

Inclusion Criteria
Subjects must have histologically or cytologically documented epithelial ovarian (FIGO Stage II-IV), fallopian tube or primary peritoneal cancer. (Subjects with pseudomyxoma or mesothelioma are excluded)
Radiographically documented progression per RECIST criteria with modifications or progression of CA-125 as defined by the Rustin during or subsequent to the last chemotherapy regimen.
May include measurable or non-measurable disease.
All scans and x-rays used to document measurable or non-measurable disease must be done within 3 weeks (21 days) of enrollment.
No more than 3 previous regimens of anti-cancer therapy. Subjects must have received at least one platinum containing regimen.
Female 18 years of age or older at the time the written informed consent is obtained.
Subjects of child-bearing potential who have not undergone a bilateral salpingo-oophorectomy and are sexually active must use an accepted and effective non-hormonal method of contraception (ie, double barrier method (eg, condom plus diaphragm)) from signing the informed consent through 6 months after last dose of study drug.
Laboratory
Adequate organ and hematological function as evidenced by the following laboratory studies within 2 weeks (14 days) of randomization:
Hematological function, as follows:
   Absolute neutrophil count (ANC)≧1.5×109/L Platelet count≧100×109/L and ≦850×109/L Hemoglobin≧9 g/dL PTT or aPTT≦1.5×ULN per institutional laboratory rand and INR≦1.5×109/L per institutional laboratory range.
   Renal function, as follows:
   Creatinine≦2.0 mg/dL Calculated creatinine clearance>40 cc/min according to the Cockcroft-Gault formula
Hepatic function, as follows:
   Total bilirubin≦2.0×ULN SGOT (AST) and SGPT (ALT)≦2.5×ULN (≦5×ULN if liver metastases are present) Nutritional
Albumin≧2.8 mg/dL General
GOG Performance Status of 0 or 1
Subject plans to begin protocol directed to therapy within 7 days of randomization.

### Exclusion Criteria

Subjects believed to be a higher than average risk for bowel perforation. This includes symptoms of partial or complete bowel obstruction, recent (within 6 months) history of fistula or bowel perforation, subjects requiring total parenteral nutrition and continuous hydration.
Known ongoing small bowel dysfunction (ie, persistent nausea, vomiting).
Radiotherapy≦14 days prior to randomization. Subjects must have recovered from all radiotherapy-related toxicities.
If all sites of disease have been irradiated, documented progression must have occurred in at least one site of disease subsequent to the radiation therapy.
Previous abdominal radiotherapy.
Has not yet completed a 21 day washout period for any previous anti-cancer systemic therapies (60 days for bevacizumab or any molecule of long half-life).
Enrolled in or has not yet completed at least 30 days since ending other investigational device or drug study(s), or is receiving other investigational agent(s).
Current or prior history of central nervous system metastasis.
Common Terminology Criteria for Adverse Events (CTCAE) Version 3.0 peripheral neuropathy≧grade 2.
History of arterial or venous thrombosis within 12 months prior to randomization.
Concurrent or prior (within 1 week before study day 1) anticoagulation therapy, excluding aspirin and anti platelet agents. The concurrent use of low molecular weight heparin or low dose warfarin (ie, ≦1 mg daily) for prophylaxis against thrombosis is acceptable while on study.
History of bleeding diathesis or clinically significant bleeding within 14 days of randomization.
Major surgical procedure within 4 weeks (28 days) prior to Study Day 1.
Minor surgical procedure, or placement of central venous access device, within 7 days of Study Day 1.
   Paracentesis and/or thoracentesis are permitted prior to and while on study at the discretion of the investigator as clinically indicated.

What is claimed:

1. A method for inhibiting the growth of ovarian epithelial carcinoma in a human patient comprising administering to said patient a therapeutically effective amount of an Ang2 inhibitor and a taxane.

2. The method of claim 1, wherein said Ang2 inhibitor is an antibody or peptibody.

3. The method of claim 2, wherein said Ang2 inhibitor is a dual Ang2 and Ang1 inhibitor.

4. The method of claim 3, wherein said peptibody is 2XCon4(C).

5. The method of claim 2, 3, or 4, wherein said taxane is paclitaxel, docetaxel, or a derivative thereof.

6. The method of claim 5, wherein said Ang2 inhibitor is administered to said patient at a dose of 3 mg/kg to about 20 mg/kg.

7. The method of claim 6, wherein said taxane is paclitaxel administered to said patient at about 80 mg/m2.

8. The method of claim 7, wherein said paclitaxel is administered once a week for three of every four weeks.

9. The method of claim 8, wherein said patient is refractory to platinum based chemotherapy.

10. The method of claim 9 wherein said platinum based chemotherapy is cisplatin, carboplatin, or oxaliplatin chemotherapy.
